SEQUENCE LISTING (22 sequences)

SEQ ID NO: 1 - protein, 434 aa, Homo sapiens
MSILKIHAREIFDSRGNPTVEVDLFTSKGLFRAAVPSGASTGIYEALELRDNDKTRYMGKGVSKAVEHINKTIAPALVSKKLNVTEQEKIDKLMIEMDGTENKSKFGANAILGVSLAVCKAGAVEKGVPLYRHIADLAGNSEVILPVPAFNVINGGSHAGNKLAMQEFMILPVGAANFREAMRIGAEVYHNLKNVIKEKYGKDATNVGDEGGFAPNILENKEGLELLKTAIGKAGYTDKVVIGMDVAASEFFRSGKYDLDFKSPDDPSRYISPDQLADLYKSFIKDYPVVSIEDPFDQDDWGAWQKFTASAGIQVVGDDLTVTNPKRIAKAVNEKSCNCLLLKVNQIGSVTESLQACKLAQANGWGVMVSHRSGETEDTFIADLVVGLCTGQIKTGAPCRSERLAKYNQLLRIEEELGSKAKFAGRNFRNPLAK

SEQ ID NO: 2 - protein, 414 aa, Homo sapiens
MAAAPPSYCFVAFPPRAKDGLVVFGKNSARPRDEVQEVVYFSAADHEPESKVECTYISIDQVPRTYAIMISRPAWLWGAEMGANEHGVCIANEAINTREPAAEIEALLGMDLVRLGLERGETAKEALDVIVSLLEEHGQGGNYFEDANSCHSFQSAYLIVDRDEAWVLETIGKYWAAEKVTEGVRCICSQLSLTTKMDAEHPELRSYAQSQGWWTGEGEFNFSEVFSPVEDHLDCGAGKDSLEKQEESITVQTMMNTLRDKASGVCIDSEFFLTTASGVSVLPQNRSSPCIHYFTGTPDPSRSIFKPFIFVDDVKLVPKTQSPCFGDDDPAKKEPRFQEKPDRRHELYKAHEWARAIIESDQEQGRKLRSTMLELEKQGLEAMEEILTSSEPLDPAEVGDLFYDCVDTEIKFFK

SEQ ID NO: 3 - protein, 490 aa, Homo sapiens
MEQKPSKVECGSDPEENSARSPDGKRKRKNGQCSLKSSMSGYIPSYLDKDEQCVVCGDKATGYHYRCITCEGCKGFFRRTIQKNLHPTYSCKYDSCCVIDKITRNQCQLCRFKKCIAVAMAMDLVLDDSKRVAKRKLIEQNRERRRKEEMIRSLQQRPEPTPEEWDLIHIATEAHRSTNAQGSHWKQRRKFLPDDIGQSPIVSMPDGDKVDLEAFSEFTKIITPAITRVVDFAKKLPMFSELPCEDQIILLKGCCMEIMSLRAAVRYDPESDTLTLSGEMAVKREQLKNGGLGVVSDAIFELGKSLSAFNLDDTEVALLQAVLLMSTDRSGLLCVDKIEKSQEAYLLAFEHYVNHRKHNIPHFWPKLLMKEREVQSSILYKGAAAEGRPGGSLGVHPEGQQLLGMHVVQGPQVRQLEQQLGEAGSLQGPVLQHQSPKSPQQRLLELLHRSGILHARAVCGEDDSSEADSPSSSEEEPEVCEDLAGNAASP

SEQ ID NO: 4 - protein, 381 aa, Homo sapiens
MPFSNSHNALKLRFPAEDEFPDLSAHNNHMAKVLTPELYAELRAKSTPSGFTLDDVIQTGVDNPGHPYIMTVGCVAGDEESYEVFKDLFDPIIEDRHGGYKPSDEHKTDLNPDNLQGGDDLDPNYVLSSRVRTGRSIRGFCLPPHCSRGERRAIEKLAVEALSSLDGDLAGRYYALKSMTEAEQQQLIDDHFLFDKPVSPLLLASGMARDWPDARGIWHNDNKTFLVWVNEEDHLRVISMQKGGNMKEVFTRFCTGLTQIETLFKSKDYEFMWNPHLGYILTCPSNLGTGLRAGVHIKLPNLGKHEKFSEVLKRLRLQKRGTGGVDTAAVGGVFDVSNADRLGFSEVELVQMVVDGVKLLIEMEQRLEQGQAIDDLMPAQK

SEQ ID NO: 5 - protein, 246 aa, Homo sapiens
VARAERQHQLYVGVLGSKLGLQVVELPADESLPDCVFVEDVAVVCEETALITRPGAPSRRKEVDMMKEALEKLQLNIVEMKDENATLDGGDVLFTGREFFVGLSKRTNQRGAEILADTFKDYAVSTVPVADGLHLKSFCSMAGPNLIAIGSSESAQKALKIMQQMSDHRYDKLTVPDDIAANCIYLNIPNKGHVLLHRTPEEYPESAKVYEKLKDHMLIPVSMSELEKVDGLLTCCSVLINKKVDS

SEQ ID NO: 6 - protein, 334 aa, Homo sapiens
MATLKEKLIAPVAEEEATVPNNKITVVGVGQVGMACAISILGKSLADELALVDVLEDKLKGEMMDLQHGSLFLQTPKIVADKDYSVTANSKIVVVTAGVRQQEGESRLNLVQRNVNVFKFIIPQIVKYSPDCIIIVVSNPVDILTYVTWKLSGLPKHRVIGSGCNLDSARFRYLMAEKLGIHPSSCHGWILGEHGDSSVAVWSGVNVAGVSLQELNPEMGTDNDSENWKEVHKMVVESAYEVIKLKGYTNWAIGLSVADLIESMLKNLSRIHPVSTMVKGMYGIENEVFLSLPCILNARGLTSVINQKLKDDEVAQLKKSADTLWDIQKDLKDL

SEQ ID NO: 7 - protein, 277 aa, Homo sapiens
MSDQQLDCALDLMRRLPPQQIEKNLSDLIDLVPSLCEDLLSSVDQPLKIARDKVVGKDYLLCDYNRDGDSYRSPWSNKYDPPLEDGAMPSARLRKLEVEANNAFDQYRDLYFEGGVSSVYLWDLDHGFAGVILIKKAGDGSKKIKGCWDSIHVVEVQEKSSGRTAHYKLTSTVMLWLQTNKSGSGTMNLGGSLTRQMEKDETVSDCSPHIANIGRLVEDMENKIRSTLNEIYFGKTKDIVNGLRSIDAIPDNQKFKQLQRELSQVLTQRQIYIQPDN

SEQ ID NO: 8 - protein, 619 aa, Homo sapiens
MSYQGKKNIPRITSDRLLIKGGKIVNDDQSFYADIYMEDGLIKQIGENLIVPGGVKTIEAHSRMVIPGGIDVHTRFQMPDQGMTSADDFFQGTKAALAGGTTMIIDHVVPEPGTSLLAAFDQWREWADSKSCCDYSLHVDISEWHKGIQEEMEALVKDHGVNSFLVYMAFKDRFQLTDCQIYEVLSVIRDIGAIAQVHAENGDIIAEEQQRILDLGITGPEGHVLSRPEEVEAEAVNRAITIANQTNCPLYITKVMSKSSAEVIAQARKKGTVVYGEPITASLGTDGSHYWSKNWAKAAAFVTSPPLSPDPTTPDFLNSLLSCGDLQVTGSAHCTFNTAQKAVGKDNFTLIPEGTNGTEERMSVIWDKAVVTGKMDENQFVAVTSTNAAKVFNLYPGAMWSAGRGGAAWPVLLGLLLALLVPGGGAAKTGAELVTCGSVLKLLNTHHRVRLHSHDIKYGSGSGQQSVTGVEASDDANSYWRIRGGSEGGCPRGSPVRCGQAVRLTHVLTGKNLHTHHFPSPLSNNQEVSAFGEDGEGDDLDLWTVRCSGQHWEREAAVRFQHVGTSVFLSVTGEQYGSPIRGQHEVHGMPSANTHNTWKAMEGIFIKPSVEPSAGHDEL

SEQ ID NO: 9 - protein, 434 aa, Homo sapiens [sequence omitted]

SEQ ID NO: 10 - protein, 364 aa, Homo sapiens
MPHSYPALSAEQKKELSDIALRIVAPGKGILAADESVGSMAKRLSQIGVENTEENRRLYRQVLFSADDRVKKCIGGVIFFHETLYQKDDNGVPFVRTIQDKGIVVGIKVDKGVVPLAGTDGETTTQGLDGLSERCAQYKKDGADFAKWRCVLKISERTPSALAILENANVLARYASICQQNGIVPIVEPEILPDGDHDLKRCQYVTEKVLAAVYKALSDHHVYLEGTLLKPNMVTPGHACPIKYTPEEIAMATVTALRRTVPPAVPGVTFLSGGQSEEEASFNLNAINRCPLPRPWALTFSYGRALQASALNAWRGQRDNAGAATEEFIKRAEVNGLAAQGKYEGSGEDGGAAAQSLYIANHAY

SEQ ID NO: 11 - protein, 335 aa, Homo sapiens
MGKVKVGVNGFGRIGRLVTRAAFNSGKVDIVAINDPFIDLNYMVYMFQYDSTHGKFHGTVKAENGKLVINGNPITIFQERDPSKIKWGDAGAEYVVESTGVFTTMEKAGAHLQGGAKRVIISAPSADAPMFVMGVNHEKYDNSLKIISNASCTTNCLAPLAKVIHDNFGIVEGLMTTVHAITATQKTVDGPSGKLWRDGRGALQNIIPASTGAAKAVGKVIPELNGKLTGMAFRVPTANVSVVDLTCRLEKPAKYDDIKKVVKQASEGPLKGILGYTEHQVVSSDFNSDTHSSTFDAGAGIALNDHFVKLISWYDNEFGYSNRVVDLMAHMASKE

SEQ ID NO: 12 - protein, 418 aa, Homo sapiens
MSLSNKLTLDKLDVKGKRVVMRVDFNVPMKNNQITNNQRIKAAVPSIKFCLDNGAKSVVLMSHLGRPDGVPMPDKYSLEPVAVELKSLLGKDVLFLKDCVGPEVEKACANPAAGSVILLENLRFHVEEEGKGKDASGNKVKAEPAKIEAFRASLSKLGDVYVNDAFGTAHRAHSSMVGVNLPQKAGGFLMKKELNYFAKALESPERPFLAILGGAKVADKIQLINNMLDKVNEMIIGGGMAFTFLKVLNNMEIGTSLFDEEGAKIVKDLMSKAEKNGVKITLPVDFVTADKFDENAKTGQATVASGIPAGWMGLDCGPESSKKYAEAVTRAKQIVWNGPVGVFEWEAFARGTKALMDEVVKATSRGCITIIGGGDTATCCAKWNTEDKVSHVSTGGGASLELLEGKVLPGVDALSNIL

SEQ ID NO: 13 - protein, 332 aa, Homo sapiens
MATLKDQLIYNLLKEEQTPQNKITVVGVGAVGMACAISILMKDLADELALVDVIEDKLKGEMMDLQHGSLFLRTPKIVSGKDYNVTANSKLVIITAGARQQEGESRLNLVQRNVNIFKFIIPNVVKYSPNCKLLIVSNPVDILTYVAWKISGFPKNRVIGSGCNLDSARFRYLMGERLGVHPLSCHGWVLGEHGDSSVPVWSGMNVAGVSLKTLHPDLGTDKDKEQWKEVHKQVVESAYEVIKLKGYTSWAIGLSVADLAESIMKNLRRVHPVSTMIKGLYGIKDDVFLSVPCILGQNGISDLVKVTLTSEEEARLKKSADTLWGIQKELQF

SEQ ID NO: 14 - protein, 260 aa, Homo sapiens
MSHHWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFK

SEQ ID NO: 15 - protein, 528 aa, Homo sapiens
MLLPLLLLLPMCWAVEVKRPRGVSLTNHHFYDESKPFTCLDGSATIPFDQVNDDYCDCKDGSDEPGTAACPNGSFHCTNTGYKPLYIPSNRVNDGVCDCCDGTDEYNSGVICENTCKEKGRKERESLQQMAEVTREGFRLKKILIEDWKKAREEKQKKLIELQAGKKSLEDQVEMLRTVKEEAEKPEREAKEQHQKLWEEQLAAAKAQQEQELAADAFKELDDDMDGTVSVTELQTHPELDTDGDGALSEAEAQALLSGDTQTDATSFYDRVWAAIRDKYRSEALPTDLPAPSAPDLTEPKEEQPPVPSSPTEEEEEEEEEEEEEAEEEEEEEDSEEAPPPLSPPQPASPAEEDKMPPYDEQTQAFIDAAQEARNKFEEAERSLKDMEESIRNLEQEISFDFGPNGEFAYLYSQCYELTTNEYVYRLCPFKLVSQKPKLGGSPTSLGTWGSWIGPDHDKFSAMKYEQGTGCWQGPNRSTTVRLLCGKETMVTSTTEPSRCEYLMELMTPAACPEPPPEAPTEDDHDEL

SEQ ID NO: 16 - protein, 266 aa, Homo sapiens
ACISFGPKNRSIGAAAKSQVISNAKNTVQGFKRFHGRAFSDPFVEAEKSNLAYDIVQLPTGLTGIKVTYMEEERNFTTEQVTAMLLSKLKETAESVLKKPVVDCVVSVPCFYTDAERRSVMDATQIAGLNCLRLMNETTAVALAYGIYKQDLPALEEKPRNVVFVDMGHSAYQVSVCAFNRGKLKVLATAFDTTLGGRKFDEVLVNHFCEEFGKKYKLDIKSKIRALLRLSQECEKLKKLMSANASDLPLSIECFMNDVDVSGTMN

SEQ ID NO: 17 - protein, 417 aa, Homo sapiens
MLLSVPLLLGLLGLAVAEPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDKEDDEDKDEDEEDEEDKEEDEEEDVPGQAKDEL

SEQ ID NO: 18 - protein, 434 aa, Homo sapiens [sequence omitted]

SEQ ID NO: 19 - protein, 445 aa, Homo sapiens
MREIVHLQAGQCGNQIGAKFWEVISDEHGIDPTGTYHGDSDLQLERINVYYNEATGGKYVPRAVLVDLEPGTMDSVRSGPFGQIFRPDNFVFGQSGAGNNWAKGHYTEGAELVDSVLDVVRKEAESCDCLQGFQLTHSLGGGTGSGMGTLLISKIREEYPDRIMNTFSVVPSPKVSDTVVEPYNATLSVHQLVENTDETYCIDNEALYDICFRTLKLTTPTYGDLNHLVSATMSGVTTCLRFPGQLNADLRKLAVNMVPFPRLHFFMPGFAPLTSRGSQQYRALTVPELTQQMFDAKNMMAACDPRHGRYLTVAAVFRGRMSMKEVDEQMLNVQNKNSSYFVEWIPNNVKTAVCDIPPRGLKMSATFIGNSTAIQELFKRISEQFTAMFRRKAFLHWYTGEGMDEMEFTEAESNMNDLVSEYQQYQDATAEEEGEFEEEAEEEVA

SEQ ID NO: 20 - protein, 340 aa, Homo sapiens
MSELEQLRQEAEQLRNQIQDARKACNDATLVQITSNMDSVGRIQMRTRRTLRGHLAKIYAMHWGYDSRLLVSASQDGKLIIWDSYTTNKMHAIPLRSSWVMTCAYAPSGNYVACGGLDNICSIYNLKTREGNVRVSRELPGHTGYLSCCRFLDDSQIVTSSGDTTCALWDIETAQQTTTFTGHSGDVMSLSLSPDMRTFVSGACDASSKLWDIRDGMCRQSFTGHVSDINAVSFFPNGYAFATGSDDATCRLFDLRADQELLLYSHDNIICGITSVAFSKSGRLLLAGYDDFNCNVWDTLKGDRAGVLAGHDNRVSCLGVTDDGMAVATGSWDSFLRIWN

SEQ ID NO: 21 - protein, 340 aa, Homo sapiens [sequence omitted]

SEQ ID NO: 22 - protein, 187 aa, Homo sapiens
MPVDLSKWSGPLSLQEVDEQPQHPLHVTYAGAAVDELGKVLTPTQVKNRPTSISWDGLDSGKLYTLVLTDPDAPSRKDPKYREWHHFLVVNMKGNDISSGTVLSDYVGSGPPKGTGLHRYVWLVYEQDRPLKCDEPILSNRSGDHRGKFKVASFRKKYELRAPVAGTCYQAEWDDYVPKLYEQLSGK

(19) United States
(12) Patent Application Publication
(10) Pub. No.: US 2009/0061465 A1
(43) Pub. Date: Mar. 5, 2009

(54) COMPOSITION FOR ANALYZING DIABETES MELLITUS AND/OR RETINAL VASCULAR DISEASE AND ANALYZING METHOD THEREOF

(75) Inventors: Yang-Je Cho, Seoul (KR); Bo-Young Ahn, Seoul (KR); Won-Il Yoo, Sungnam (KR); Oh-Woong Kwon, Goyang (KR)

Correspondence Address: EDWARDS ANGELL PALMER & DODGE LLP, P.O. BOX 55874, BOSTON, MA, 02205, US

(73) Assignee: EYEGENE INC., Seoul (KR)

(21) Appl. No.: 11/631,892

(22) PCT Filed: Jul. 6, 2005
(86) PCT No.: PCT/KR2005/002171

§ 371 Date: Oct. 10, 2008

(30) Foreign Application Priority Data
Jul. 6, 2004 (KR) 10-2004-0052385

(51) Int. Cl. G01N 33/53

(52) U.S. Cl. 435/7.92; 435/7.1

(57) ABSTRACT

Disclosed are composition for diagnosing a diabetes mellitus and a retinal vascular disease, a kit for diagnosing the retinal vascular disease including the protein, a gene encoding the protein, and a method for analyzing an antibody prepared by the diabetes mellitus and/or the retinal vascular disease using the same.

### TECHNICAL FIELD

The present invention relates to composition for diagnosing a diabetes mellitus and a retinal vascular disease, a kit for diagnosing the retinal vascular disease including the protein, a gene encoding the protein, and a method for analyzing antibodies prepared by the diabetes mellitus and/or the retinal vascular disease using the same.

### BACKGROUND ART

Generally, a diabetes mellitus has been known as a complex metabolic disease that induces lesions in a microvascular system. The diabetes mellitus is one of the most important systemic disorders which leads to wide ranges of disorders in systemic tissues, and particularly have an effect on eyes (Tae-Hee, Lee and Young-Gil, Choi, Diabetic Vascular Complication, Seoul of Republic of Korea, Korea Medical Book publisher, 1993). It was known that the diabetic retinopathy belongs to one of the most serious complications of the diabetes mellitus, and has been prevalent as a social problem since life span and illness duration of the diabetics are increasingly prolonged with improvement of the living standard and development of medical cures (Klein R. et al., Arch Opthalmol., 102, 520-532, 1984). The diabetic retinopathy is divided into two groups: one is a non-proliferative diabetes retinosis (NPDR) in which retinal lesions by vascular disorders is defined in the retina, and the other is a proliferative diabetes retinosis (PDR) in which angiogenetic tissues are infiltrated from the retina to the vitreous cavity (Green, In: Spencer WH, ed., Ophthalmic Pathology: an atlas and textbook. 4th ed., Philadelphia: WB Saunder; 1124-1129, 1996). In the proliferative diabetes retinosis, damage of eyesight by the diabetic retinopathy is derived from a vitreous hemorrhage, a traction retinal detachment of a macula lutea and a macular degeneration, and surgery and laser treatments are well known to be effective for treatment of the above-mentioned diseases (Diabetic Retinopathy Study Report Number 14, Int Opthalmol Clin., 27, 239-253, 1987). Conducting such a treatment at a suitably advanced stage of the above-mentioned diseases makes it possible to minimize side effects of the diabetes mellitus and also prevent loss of eyesight. Accordingly, diagnosis of the diabetic retinopathy should be periodically conducted to secure suitable time for the surgery.

The diabetes mellitus may be diagnosed by measuring an amount of sugars in urine or blood, but the measured amount of sugars may be varied depending on intaked meals and others and make it impossible to diagnose the progress of the diseases.

The diabetic retinopathy has been diagnosed by observing characteristic structural changes in the ocular fundus, of which diagnosis has been conducted only through ocular inspection by opthalmography in ophthahnic hospitals. Accordingly, it is possible to diagnose the diabetic retinopathy at an early stage only if the diabetics feels a subjective symptom such as an visual disorder and undergoes periodical ophthalmologist examinations. As a result, it is difficult to diagnose the diabetic retinopathy at an early stage, and therefore the patients may often miss an opportunity for prevention and surgical operation of the diabetic retinopathy.

### DISCLOSURE

### Technical Problem

Accordingly, the present invention is designed to solve the problems of the prior art, and therefore it is an object of the present invention to provide composition for diagnosing a diabetes mellitus, including at least one protein selected from the group consisting of proteins set forth in amino acid sequences of SEQ ID NOs: 1 to 22, or fragments thereof.

Also, it is another object of the present invention to provide composition for diagnosing a vascular diseases, including at least one protein selected from the group consisting of proteins set forth in amino acid sequences of SEQ ID NOs: 1 to 9 and SEQ ID NOs: 11 to 22, or fragments thereof.

Preferably, the composition of the present invention includes a protein or its fragments set forth in an amino acid sequence of SEQ ID NO: 4.

In the present invention, the retinal vascular disease includes, but is not limited to, a diabetic retinopathy, a retinal edema and an age-related macular degeneration.

Also, it is still another object of the present invention to provide a kit for diagnosing a diabetes mellitus or a retinal vascular disease, including the protein or its fragments of the present invention.

Also, it is still another object of the present invention to provide a kit for diagnosing a retinal vascular disease, including the protein or its fragments of the present invention. In the present invention, the kit preferably further includes a labeled anti-immunoglobulin G antibody protein.

Also, it is still another object of the present invention to provide a method of analyzing the antibody generated by the retinal vascular disease, including a step of contacting with blood at least one protein at least one protein selected from the group consisting of proteins set forth in amino acid sequences of SEQ ID NOs: 1 to 9 and SEQ ID NOs: 11 to 22. In the present invention, the method of analyzing the antibody preferably further includes a step of adding a labeled anti-immunoglobulin G antibody protein.

### Technical Solution

Hereinafter, the present invention will be described, as follows.

In order to satisfy the above requirements, the present invention provides a protein and its fragments for diagnosing a diabetes mellitus and a retinal vascular disease, a kit for diagnosing the retinal vascular disease including the protein and its fragments, a gene encoding the protein and its fragments, and a method for analyzing an antibody generated by the retinal vascular disease using the same.

In order to accomplish the above objects, the present invention provides a protein and its fragments for diagnosing a diabetes mellitus or diseases, a diagnostic kit including the protein and its fragments, a gene encoding the protein and its fragments, and a method for analyzing an antibody generated by the retinal vascular disease using the same.

The inventors found that since blood-ocular barriers are present in blood vessels of eyeball, retinal proteins are not exposed to an immune system under a normal state, but exposed to an immune system to generate autoantibodies in the case of the diseases such as a diabetes mellitus. Also, the inventors found a diagnostic protein and its fragments for easily diagnosing the retinal vascular disease in blood using the autoantibody, a kit including the protein and its fragments, a gene encoding the protein and its fragments, and a method for analyzing an antibody generated by a diabetes mellitus using the same.

Hereinafter, preferred embodiments of the present invention will be described in detail.

In order to accomplish the above objects, one embodiment of the present invention provides a protein and its fragments for diagnosing a retinal vascular disease, the protein and its fragments consisting of amino acid sequences of SEQ ID NOs: 1 to 9 and SEQ ID NOs: 11 to 22. The inventors found that the protein and its fragments of the present invention may be hemorrhaged from retinal blood vessels of a patient suffering from the retinal vascular disease to induce autoantibodies in blood, and therefore the increased autoantibodies may be measured using the protein and its fragments of the present invention.

In order to confirm the above-mentioned result, experiments such as one- and two-dimensional western immunoblotting were conducted by the inventors. Also, an experiment for analyzing patient's blood was conducted by an enzyme-linked immunosorbent assay (hereinafter, it is referred to as “ELISA”) using some of various proteins capable of producing autoantibodies. As a result, it was confirmed that the method according to the present invention may effectively diagnose the retinal vascular disease.

The proteins include proteins derived from humans, as well as animals.

In amino acid sequences of the proteins according to the present invention, at least one amino acid may be substituted, added and deleted without having an effect on functions of the protein, and fragments of the proteins may be also used, if necessary. Such a modified amino acid sequence is also included in the scope of the present invention. Accordingly, the protein of the present invention also includes polypeptides having substantially identical amino acid sequences to that of the retinal vascular diseases. The term “substantially identical polypeptide” is meant that polypeptides have sequence homology of at least at least 80%, preferably at least 90%, and most preferably at least 95%.

The disease which may be diagnosed by the method of the present invention are, but not limited to, preferably a disease whose retinal proteins are exposed to the blood vessels of eyeball, for example a diabetes mellitus, a diabetic retinopathy, an age-related macular degeneration, a retinal edema, etc. and especially preferably a diabetic retinopathy.

Another embodiment of the present invention provides a diagnostic kit including a protein or its fragments. The kit of the present invention may be used in the ELISA method including the protein or its fragments, and various additives may also be included in the kit of the present invention, as is apparent to those skilled in the art pertaining to the present invention. Preferably, the kit of the present invention has a well plate to which the protein or its fragments are attached.

Preferably, the diagnostic kit of the present invention also includes anti-antibody proteins capable of attaching to the autoantibodies prepared in the blood of the patient by the protein or its fragments, and more preferably includes a label for measuring its absorbance. The anti-antibody protein may be labeled with various enzymes available in the ELISA method, such as peroxydase, alkaline phosphatase, biotin, etc., as is apparent to those skilled in the art pertaining to the present invention.

Still another embodiment of the present invention provides a gene encoding the protein for diagnosing the diabetic disease, the protein consisting of amino acid sequences of SEQ ID NOs: 1 to 9 and SEQ ID NOs: 11 to 22. Due to degeneracy of a codon, or considering a codon preferential in living things designed to express the protein, various changes and modifications may be made in a coding region of the gene of the present invention without modifying the amino acid sequence of the desired protein for diagnosing the diabetic disease, the desired protein expressed from the coding region, and various changes and modifications may also be made only in a region except the coding region without having an effect on expression of the gene. Such a modified gene is also included in the scope of the present invention. Accordingly, the gene of the present invention also includes polynucleotides having the substantially identical base sequence to that of the gene encoding the protein. The term “substantially identical polynucleotide” is meant that polynucleotides have base sequence homology of at least at least 80%, preferably at least 90%, and most preferably at least 95%.

Yet another embodiment of the present invention provides a method for analyzing an antibody generated by diabetic disease, including a step of contacting the diagnostic protein with collected bloody. Preferably, the method for analyzing the antibody further includes a step of adding the labeled anti-antibody protein. More preferably, the anti-antibody protein is an anti-immunoglobulin G antibody protein.

Also in the method for analyzing antibody, the label is preferably detected by using absorbance. The label includes various enzyme, which may be used in ELISA assay, such as peroxydase, alkaline phosphatase, biotin, etc., as is apparent to those skilled in the art pertaining to the present invention.

### DESCRIPTION OF DRAWINGS

FIG. 1 is a photograph showing western blots of human retinal proteins for cytosol fractions and membrane fractions of a healthy human, diabetics, a patient suffering from non-proliferative diabetes retinosis, a patient suffering from proliferative diabetes retinosis.

FIG. 2 is a photograph showing two-dimensional electrophoresis of the human retinal protein.

FIG. 3 is a photograph in which the electrophoresed gel of FIG. 2 is divided into four parts and western-blotted using serum of a healthy male.

FIG. 4 shows a photograph in which the electrophoresed gel of FIG. 2 is divided into four parts and western-blotted using serum of a patient suffering from a non-proliferative diabetes retinosis.

FIG. 5 shows a photograph in which the electrophoresed gel of FIG. 2 is divided into four parts and western-blotted using serum of a patient suffering from a proliferative diabetes retinosis.

FIG. 6 is a diagram showing a result in which serums of a healthy human, diabetics, a patient suffering from a non-proliferative diabetes retinosis, a patient suffering from a proliferative diabetes retinosis are diagnosed by means of ELISA using creatine kinase B.

FIG. 7 is a diagram showing a result in which serums of a healthy human, diabetics, a patient suffering from a non-proliferative diabetes retinosis, a patient suffering from a proliferative diabetes retinosis are diagnosed by means of ELISA using aldolase.

### BEST MODE

Hereinafter, preferred embodiments of the present invention will be described in detail referring to the accompanying drawings. Therefore, the description proposed herein is just a preferable example for the purpose of illustrations only, not intended to limit the scope of the invention, so it should be understood that other equivalents and modifications could be made thereto without departing from the spirit and scope of the invention.

### Example 1

### Analysis of Autoantibodies on Human Retinal Protein Using Western Blot

Separation of Human Retinal Protein

Only a retina was extracted from an eyeball, and then washed with saline several times. A cytosol fraction and a membrane fraction were separated using a ProteoPrep Universal Extraction Kit (Sigma S2813), and then quantitified using a Pierce BCA Protein Assay Kit 23227 (Pierce, U.S.).

Western Blotting of Retinal Protein on Serums of Healthy Human and Patients

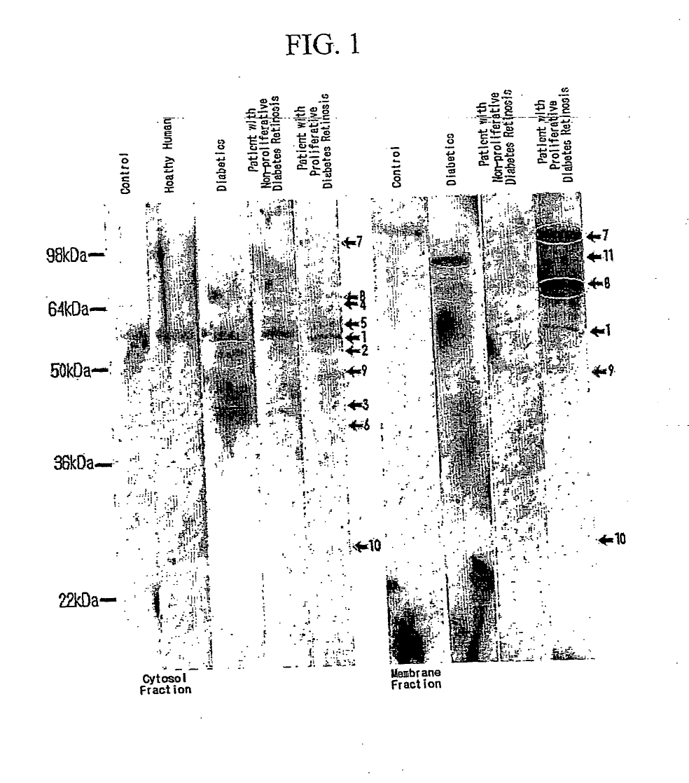

30 μg of retinal protein was electrophoresed in 12% acrylamide gel, and the electrophoresed acrylamide gel was then transferred into a nitrocellulose membrane and western-blotted using serums of a healthy human, diabetics, a patient suffering from a non-proliferative diabetes retinosis and a patient suffering from a proliferative diabetes retinosis to confirm weather the autoantibody is generated. The result is summed up and listed in FIG. 1 and Table 1.

TABLE 1

| IgG | Molecular | Cytosol Fraction | | | | | | | | |
|---|---|---|---|---|---|---|---|---|---|---|
| Heavy | Weight | | healthy | | | Membrane Fraction | | | | |
| Chain | (kDa) | Control | human | DM | NPDR | PDR | Control | DM | NPDR | PDR |
| 1 | 56.1 | + | + | + | + | + | | | | + |
| 2 | 53.4 | | | ++ | + | + | | | | |
| 3 | 44.4 | + | + | +++ | ++ | + | | | | |
| 4 | 63.5 | | | | + | | | | | |
| 5 | 58.9 | | | | + | + | | | | |
| 6 | 40.7 | | | | | + | | | | |
| 7 | 79.2 | | | | | + | | | | + |
| 8 | 65.0 | | | | | + | | + | + | + |
| 9 | 49.6 | | | + | | ++ | | | + | + |
| 10 | 26.1 | | | | | + | | | | + |
| 11 | 73.6 | | | | | | | +++ | | |

In Table 1, DM represents diabetics, NPDR represents a patient suffering from a non-proliferative diabetes retinosis, PDR represents a patient suffering from a proliferative diabetes retinosis. “+” represents that positive band is observed, and its number represents intensity of the band.

### Example 2

### Two-Dimensional Gel Western Blotting of Human Retinal Protein

Two-Dimensional Gel Electrophoresis

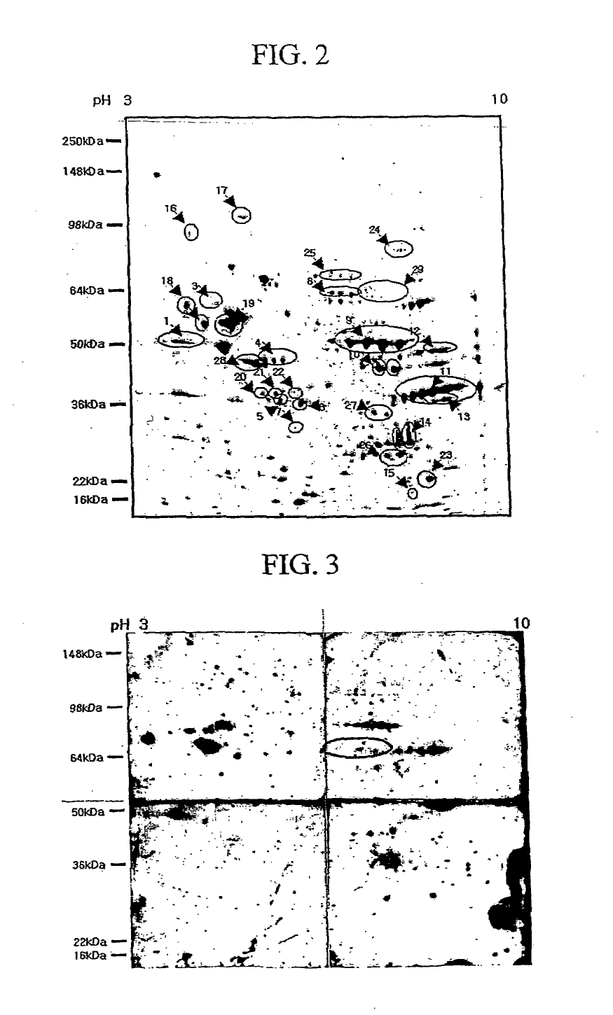

A retinal protein was separated by successive separation such as a two-dimensional gel electrophoresis using two different properties of the retinal protein. The two-dimensional gel electrophoresis was carried out in two steps: a first step is migrating proteins depending on their pH when electrical stimulus is applied to the proteins (pH 3-10), and a second step is migrating the proteins on acrylamide gel (8-18%) depending on their molecular weights. One-dimensional electrophoresis (or, migration of the proteins depending on pH) was conducted for 12 hours at an electric current of 50 mA per a gel, and two-dimensional electrophoresis (or, migration of the proteins depending on the molecular weight) was conducted on a polyacrylamide gel for 6 ours at an electric current of 50 mA per a gel. It was confirmed whether the desired protein was present by dying the migrated proteins using a Coomassie Brilliant Blue-250 dye and a silver staining method. Four gels were obtained in the same manner. One gel was used to confirm 2-dimensional gel distribution of the proteins derived from a healthy human, and each of the other three gels were divided into four parts and used to conduct the western blotting. The results are shown in FIG. 2. The numbers shown in FIG. 2 represent spot numbers of following Table 2.

Western Blotting

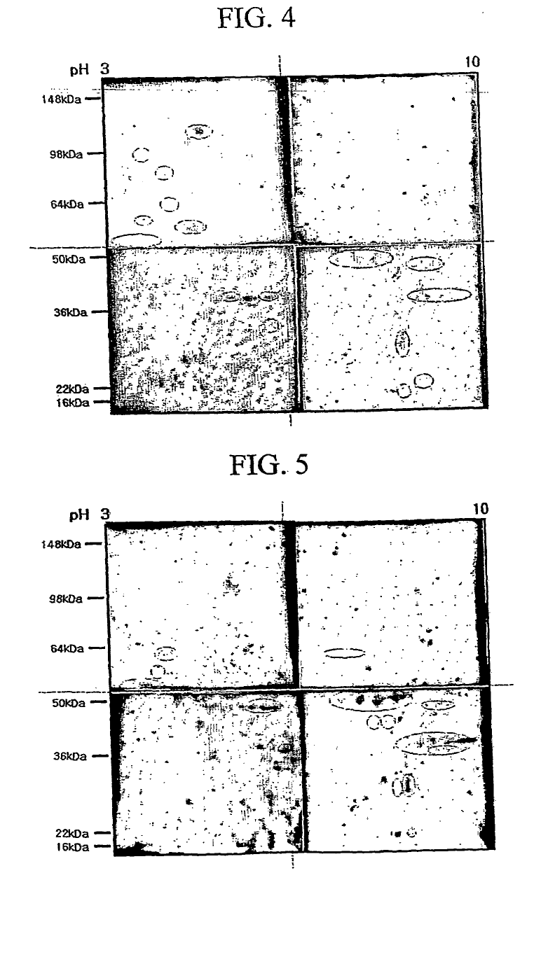

The gel obtained in the two-dimensional gel Electrophoresis was blotted to confirm a location of the autoantibody using serums of a healthy human, a patient suffering from a non-proliferative diabetes retinosis and a patient suffering from a proliferative diabetes retinosis. The results are shown in FIGS. 3, 4 and 5.

Difference between antibodies derived from the healthy humans and the patients suffering from the diabetic retinopathy was analyzed with an Image-analyzing software Phoretix (Nonlinear dynamics, the United Kingdom) using a computer. It was confirmed that spots, which were obtained by matching the result analyzed between two groups to the two-dimensional gel, were analyzed using MALDI-TOF, and the autoantibody against the proteins of Table 1 were formed in blood of the patient suffering from the diabetic retinopathy. Antigen proteins against the autoantibody present in the patient suffering from the diabetic retinopathy are summed up and listed in Table 2.

TABLE 2

| Spot No. | | Name of Protein |
|---|---|---|
| 1 | (SEQ ID NO: 1) | Alpha enolase, non-neural enolase |
| 2 | (SEQ ID NO: 2) | Protein KIAA0193 |
| 3 | (SEQ ID NO: 3) | Unnamed protein product thyroid hormone |
| | | binding protein precursor |
| 4 | (SEQ ID NO: 4) | Creatine kinase-B |
| 5 | (SEQ ID NO: 5) | DDAH1 protein |
| 6 | (SEQ ID NO: 6) | Lactate dehydrogenase B |
| 7 | (SEQ ID NO: 7) | Capping protein (actin filament)) muscle z- |
| | | line, beta |
| 8 | (SEQ ID NO: 8) | Dihydropyrimidinase-like 2 |
| 9 | (SEQ ID NO: 9) | 2-phosphopyruvate-hydratase alpha-enolase |
| 10 | (SEQ ID NO: 10) | Aldolase C |
| 11 | (SEQ ID NO: 11) | Glyceraldehyde-3-phosphate dehydrogenase |
| 12 | (SEQ ID NO: 12) | Phosphoglycerate kinase 1 (primer recognition |
| | | protein 2), (PRP2) |
| 13 | (SEQ ID NO: 13) | Lactate dehydrogenase A |
| 14 | (SEQ ID NO: 14) | Carbonic anhydrase II |
| 15 | (SEQ ID NO: 15) | Glucosidase II beta subunit |
| 16 | (SEQ ID NO: 16) | HS24/P52 |
| 17 | (SEQ ID NO: 17) | Calreticulin |
| 18 | (SEQ ID NO: 19) | Tubulin beta-4q chain |
| 19 | (SEQ ID NO: 19) | Beta-tubulin |
| 20 | (SEQ ID NO: 20) | Guanine nucleotide-binding protein, beta-4 |
| 21 | (SEQ ID NO: 21) | Guanine nucleotide-binding protein (G protein), |
| | | beta polypeptide 1 |
| 22 | (SEQ ID NO: 22) | Prostatic binding protein; phosphatidylethanol- |
| | | amine binding protein |

### Example 3

### Diagnosis of Diabetic Retinopathy by ELISA Using Creatine Kinase B

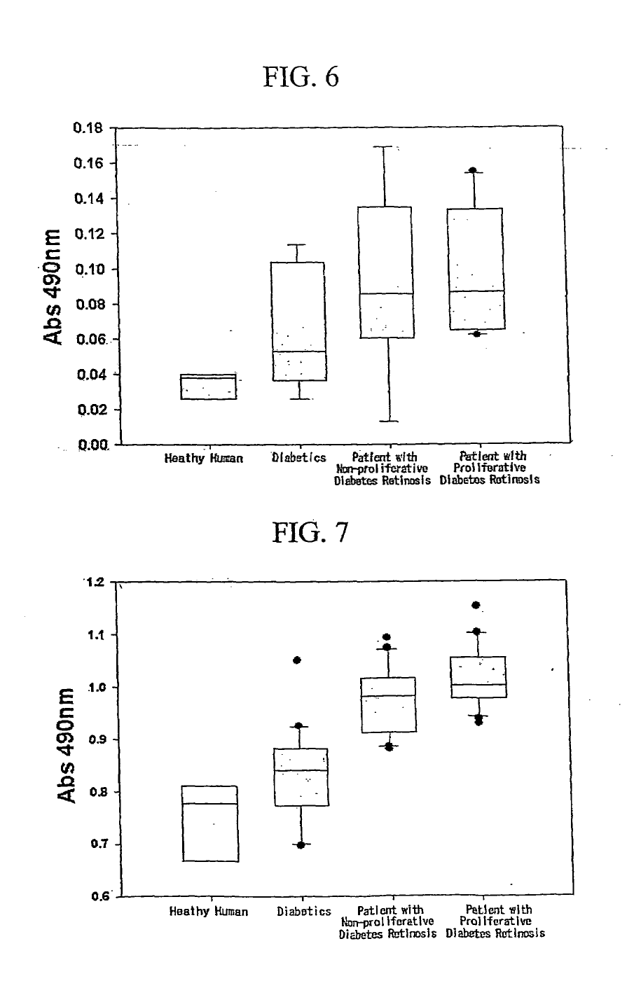

This experiment was carried out to confirm whether serums of patients suffering from the diabetic retinopathy are distinguished from those of the diabetics by means of ELISA using creatine kinase B. Serums of three healthy humans, ten diabetics which does not suffer from the diabetic retinopathy, and twenty patients suffering from the diabetic retinopathy were used in this experiment, the serums kindly provided by a hospital. At first, a 96-well EIA plate was coated at a room temperature for 1 hour with 100 μl of creatine kinase B (Sigma, C6638) (1 μg of the protein per a well) which is dissolved in a coating buffer (50 mM NaHCO₃, pH 9.0) at a concentration of 10 μg/Ml per a well, washed two times (each 10 minutes) with 400 μl of PBST (phospate buffer saline, 0.05% Tween 20), and then post-coated with PBS containing 1% BSA (bovine serum albumin). 100 μl of the patient's serum diluted with PBST was added thereto, reacted for 1 hour, and washed five times with PBS. 100 μl of an peroxydase-labeled anti-human immunoglobulin G antibody (KOMA Biotech Inc., Republic of Korea) was diluted and added to the resultant serum, and the resultant mixture was reacted for 1 hour. After the reaction was completed, the mixture was washed three times with PBS, and 100 μl of 0.1 M citrate-phosphate buffer (pH 4.9) containing 1 mg/Ml OPD (o-phenylenediamine dihydrochloride) and 0.03% H₂O₂ was added thereto and reacted for 30 minutes at a room temperature, and then the reaction was finished by adding 100 μl of 3 M sulfuric acid to the reaction mixture. Absorbance of the resultant reaction mixture was measured at 450 nm using an ELISA reader. The result is shown in FIG. 6. As a result of ELISA, mean values of creatine kinase B in the serum were 0.04 for healthy humans, 0.06 for patients suffering from only the diabetes mellitus, 0.08 for patients suffering from the non-proliferative diabetic retinopathy, and 0.08 for patients suffering from the proliferative diabetic retinopathy. From the above result, it was confirmed that the amount of creatine kinase B was increased in serums of the patients suffering from the non-proliferative and proliferative diabetes retinosis.

### Example 4

### Diagnosis of Diabetes Mellitus by ELISA Using Creatine Kinase B

Also, serums of 30 healthy humans and 60 diabetics were used in this experiment, the serums kindly provided by any hospitals. And this experiment was carried out by means of ELISA in the same manner as in Example 3. The result was listed in Table 3. It was revealed that creatine kinase B may be used to diagnoses the diabetes mellitus.

TABLE 3

| | Healthy human | Diabetics |
|---|---|---|
| 0.05 or more | 5 | 55 |
| 0.05 or less | 25 | 5 |

### Example 5

### Diagnosis of Diabetic Retinopathy by ELISA Using Aldolase

This experiment was carried out to confirm whether serums of patients suffering from the diabetic retinopathy are distinguished from that of the diabetics by means of ELISA using aldolase. Serums of three healthy humans, ten diabetics which does not suffer from the diabetic retinopathy, and twenty patients suffering from the diabetic retinopathy were used in this experiment, the serums kindly provided by a hospital. At first, a 96-well EIA plate was coated at a room temperature for 1 hour with 100 μl of aldolase (Sigma, A2714) (1 μg of the protein per a well) which is dissolved in a coating buffer (50 mM NaHCO₃, pH 9.0) at a concentration of 10 μg/Ml per a well, washed two times (each 10 minutes) with 400 μl of PBST, and then post-coated with PBS containing 1% BSA. 100 μl of the patient's serum diluted with PBST was added thereto, reacted for 1 hour, and washed five times with PBS. 100 μl of an peroxydase-labeled anti-human immunoglobulin G antibody (KOMA Biotech Inc., Republic of Korea) was diluted and added to the resultant serum, and the resultant mixture was reacted for 1 hour. After the reaction was completed, the mixture was washed three times with PBS, and 100 μl of 0.1 M citrate-phosphate buffer (pH 4.9) containing 1 mg/Ml OPD and 0.03% H₂O₂ was added thereto and reacted for 30 minutes at a room temperature, and then the reaction was finished by adding 100 μl of 3 M sulfuric acid to the reaction mixture. Absorbance of the resultant reaction mixture was measured at 450 nm using an ELISA reader. The result is shown in FIG. 7. As a result of ELISA, mean values of aldolase C in the serum were 0.78 for healthy humans, 0.84 for patients suffering from only the diabetes mellitus, 0.98 for patients suffering from the non-proliferative diabetic retinopathy, and 1.0 for patients suffering from the proliferative diabetic retinopathy. From the above result, it was confirmed that the amount of aldolase C was increased in the patients suffering from the non-proliferative and proliferative diabetes retinosis.

As seen from the results of Examples 3 and 5, the amount of creatine kinase B was increased by 50% for the non-proliferative and the proliferative diabetes retinosis, respectively, and the amount of aldolase C was increased by 25% and 28% for the non-proliferative and the proliferative diabetes retinosis, respectively, compared to the healthy humans.

From the above result, it is confirmed that the patients may be diagnosed to suffer from the diabetic retinopathy if they have the increased amount of serum in blood.

### Example 6

### Diagnosis of Diabetes Mellitus by ELISA Using Aldolase C

Also, serums of 30 healthy humans and 60 diabetics were used in this experiment, the serums kindly provided by any hospitals. And this experiment was carried out by means of ELISA in the same manner as in Examples 5. The result was listed in Table 4. It was revealed that aldolase C may be used to diagnoses the diabetes mellitus.

TABLE 4

| | Healthy human | Diabetics |
|---|---|---|
| 0.8 or more | 6 | 56 |
| 0.8 or less | 24 | 4 |

### INDUSTRIAL APPLICABILITY

According to the present invention, the protein for diagnosing the diabetes and the vascular diseases, the kit including the protein, and the analyzing method using the same may be useful to diagnose the diseases simply and rapidly, and they also may show excellent accuracy and precision, compared to the conventional diagnostic methods. According to the present invention, the kit for diagnosing the diabetes and the vascular disease, and the analyzing method using the same are also very economical.

1. A composition for diagnosing diabetes mellitus, comprising at least one protein selected from the group consisting of proteins having the amino acid sequences represented by SEQ ID NOs: 1 to 22, or fragments thereof.

2. A composition for diagnosing vascular diseases, comprising at least one protein selected from the group consisting of proteins having the amino acid sequences represented by SEQ ID NOs: 1 to 9 and SEQ ID NOs: 11 to 22, or fragments thereof.

3. The composition according to claim 1,
wherein the composition comprises a protein having the amino acid sequence represented by SEQ ID NO: 4, or fragments thereof.

4. The composition according to the claim 2,
wherein the vascular disease is selected from the group consisting of diabetic retinopathy, retinal edema and age-related macular degeneration.

5. A kit for diagnosing diabetes mellitus, comprising the protein defined in claim 1, or fragments thereof.

6. A kit for diagnosing vascular diseases, comprising the protein defined in claim 2, or fragments thereof.

7. The kit according to claim 5,
further comprising a labeled anti-immunoglobulin G antibody protein.

8. A method of analyzing an antibody generated by diabetes mellitus or vascular diseases, comprising a step of contacting blood with at least one protein defined in claim 1.

9. The method according to claim 8,
further comprising a step of adding a labeled anti-immunoglobulin G antibody protein.

10. The composition according to claim 2,
wherein the composition comprises a protein having the amino acid sequence represented by SEQ ID NO: 4, or fragments thereof.

11. The kit according to claim 6, further comprising a labeled anti-immunoglobulin G antibody protein.

12. A method of analyzing an antibody generated by diabetes mellitus or vascular diseases, comprising a step of contacting blood with at least one protein defined in claim 2.

13. The method according to claim 12, further comprising a step of adding a labeled anti-immunoglobulin G antibody protein.
